(19) United States
(12) Patent Application Publication
(10) Pub. No.: US 2025/0375619 A1
(43) Pub. Date: Dec. 11, 2025

(54) MULTIFUNCTIONAL BELT

(71) Applicant: Yin LIU, Shenzhen (CN)

(72) Inventors: Yin LIU, Shenzhen (CN); Caiyun GAO, Shenzhen (CN)

(21) Appl. No.: 18/914,325

(22) Filed: Oct. 14, 2024

(30) Foreign Application Priority Data
Jun. 6, 2024 (CN) 202410729061.5

(51) Int. Cl. A61N 2/00; A61H 23/02; A61N 5/06

(52) U.S. Cl. CPC A61N 2/002; A61H 23/0254; A61N 5/0613; A61H 2201/0157; A61H 2201/0228; A61H 2201/10; A61H 2201/165; A61N 2005/0645; A61N 2005/0652; A61N 2005/0664

(57) ABSTRACT

The invention discloses a multifunctional belt, which comprises a belt surface layer and a belt inner layer, wherein the belt surface layer is buckled on the belt inner layer, the belt surface layer is provided with a battery, a PCB board and an LED light panel, the battery is connected to the PCB board through a wire, the LED light panel is provided with an LED light, the belt inner layer is provided with a convex lens, and the LED light emits a light beam to be radiated onto the skin through the convex lens, the multifunctional belt can perform phototherapy on the human body and focus the light beam on the deep skin during phototherapy to improve the phototherapy effect, and the product is thin and portable during use.

### TECHNICAL FIELD

The invention relates to the technical field of massage belts, in particular to a multifunctional belt.

### BACKGROUND ART

Massage belt is a common massage appliance. With the development of the society and the improvement of people's living standards, it is becoming more and more common for people to relieve fatigue through physiotherapy products. The massage belt can massage the waist, back and legs to relieve muscle pain and promote blood circulation in the waist. As a convenient and efficient massage tool, the massage belt has been widely used. The belts in the prior art have many technical problems such as large volume, heavy weight, inconvenient use and portability, and poor effect of focusing light to the surface of the skin during phototherapy.

### SUMMARY OF THE INVENTION

Aiming at the prior art, the invention provides a multifunctional belt, which can perform phototherapy on the human body and focus the light beam on the deep skin during phototherapy to improve the phototherapy effect, and the product is thin and portable during use.

To achieve the above purpose, the technical proposal of the embodiments of the invention is realized as follows:

A multifunctional belt comprises a belt surface layer and a belt inner layer, wherein the belt surface layer is buckled on the belt inner layer, the belt surface layer is provided with a battery, a PCB board and an LED light panel, the battery is connected to the PCB board through a wire, the LED light panel is provided with an LED light, the belt inner layer is provided with a convex lens, and the LED light emits a light beam to be radiated onto the skin through the convex lens.

Further, the belt inner layer is provided with a base, and the base is provided with a vibrating motor.

Further, the base is provided with a magnet.

Further, the base is provided with a stainless steel cap, the stainless steel cap is covered on the base, the base is provided with a PCBA board, the PCBA board is provided with a resistor.

Further, the PCBA board is provided with an EMS conducting area.

Further, the belt surface layer is provided with a grille, on which the LED light panel is installed.

Further, the belt surface layer and the belt inner layer are made of silicone.

Further, the base is provided with a motor hole and a magnet hole, and the vibrating motor is installed on the motor hole, and the magnet is installed in the magnet hole.

Further, the belt surface layer is provided with the battery and the PCB board, and the battery is electrically connected with the PCB board, and the belt surface layer is also provided with a power switch, a heating switch, an EMS switch, an indicator light and a charging port, all of which are connected with the PCB board through wires.

Further, a silicone thermal conductive pad is arranged on the resistor.

The invention has the following beneficial effects: (1) a convex lens is arranged in the belt inner layer, a light beam is emitted through an LED light and focused on the deep layer of the skin through the convex lens, thus improving the phototherapy effect; (2) The multifunctional belt integrates light therapy, heat therapy, vibration massage and magnetic health care into a whole, and utilizes space reasonably, thus reducing the weight; (3) The product is thin, light and portable.

### BRIEF DESCRIPTION OF THE DRAWINGS

FIG. 1 is the assembly diagram for a multifunctional belt in the embodiment of the application.

FIG. 2 is the explosive view for a multifunctional belt in the embodiment of the application.

FIG. 3 is the structural diagram for a belt surface layer in the embodiment of the application.

FIG. 4 is the enlarged view for point A in the embodiment of the application.

FIG. 5 is the structural diagram for a belt inner layer in the embodiment of the application.

FIG. 6 is the enlarged view for point B in the embodiment of the application.

FIG. 7 is the explosive view for a base in the embodiment of the application.

FIG. 8 is the schematic diagram for parts of a belt surface layer and a belt inner layer in the embodiment of the application.

### DESCRIPTION OF REFERENCE SIGNS

1. Belt surface layer; 2. Belt inner layer; 3. LED light board; 4. LED light; 5. Convex lens; 6. Base; 601. Stainless steel cap; 7. Vibrating motor; 8. Magnet; 9. PCBA board; 10. Resistor; 11. EMS conducting area; 12. Grille; 14. Motor hole; 15. Magnet hole; 16. PCB board; 17. Power switch; 18. Heating switch; 19. EMS switch; 20. Indicator light; 21. Charging port; 22. Gland; 23. Battery; 24. Silicone thermal conductive pad; 25. Bulge; 26. Notch.

### DETAILED DESCRIPTION OF THE INVENTION

Unless otherwise defined, all technical and scientific terms used herein have the same meaning as that normally understood by the technicians in the technical field of the invention. The terms used in the specification of the invention are for the purpose of describing the embodiments only and are not intended to limit the invention. In the following description, reference to the expression “some embodiments” describes a subset of all possible embodiments. However, it should be understood that “some embodiments” may be the same or different subsets of all possible embodiments and may be combined without conflict.

It should also be noted that a component can be directly located on the other component or an intermediate component exists when the same component is said to be “fixed” to another component. When a component is considered to be “connected” with another component, the same component can be directly connected to another component or an intermediate component exists. The terms “vertical”, “horizontal”, “inside”, “outside”, “left” and “right” and similar expressions used herein are for explanatory purpose only and are not intended to represent the sole embodiment.

### Embodiment 1

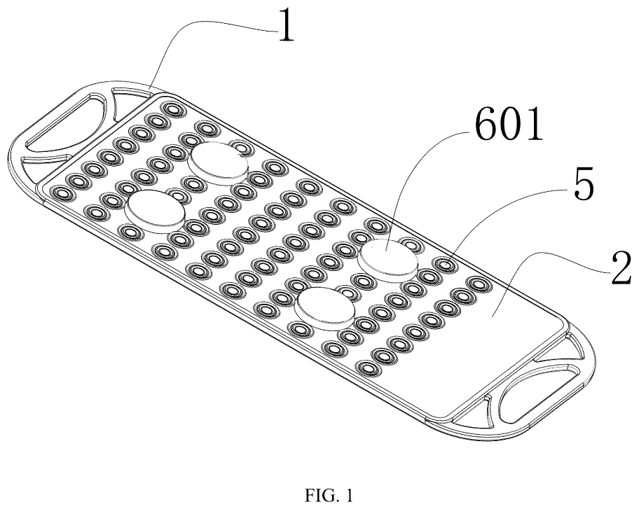

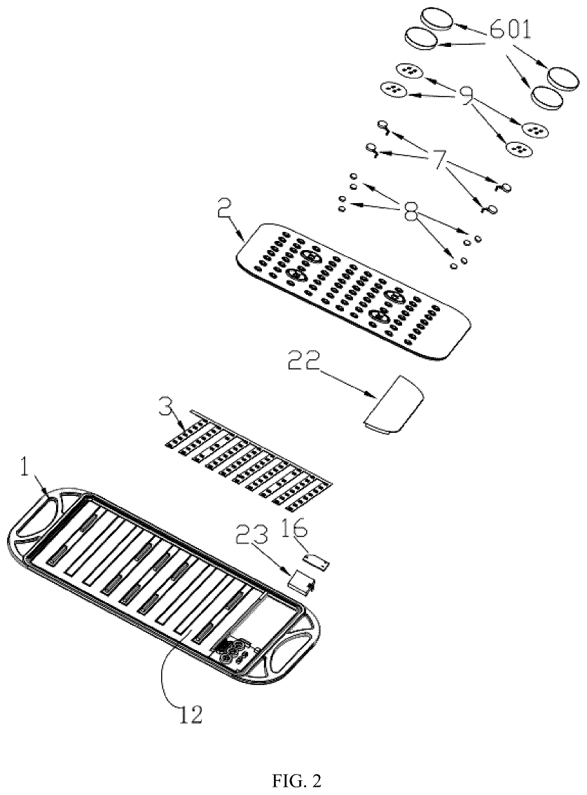

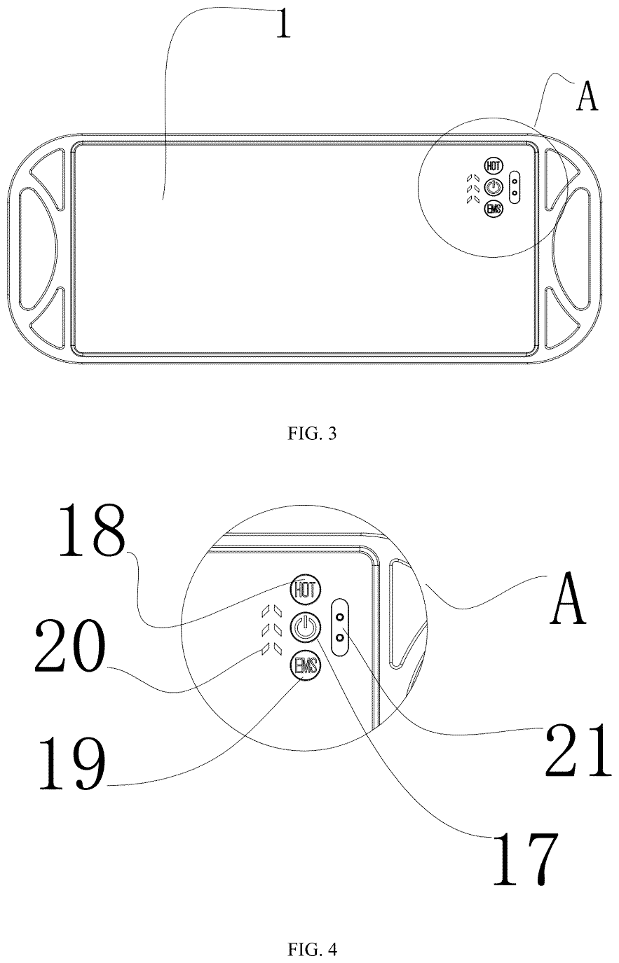

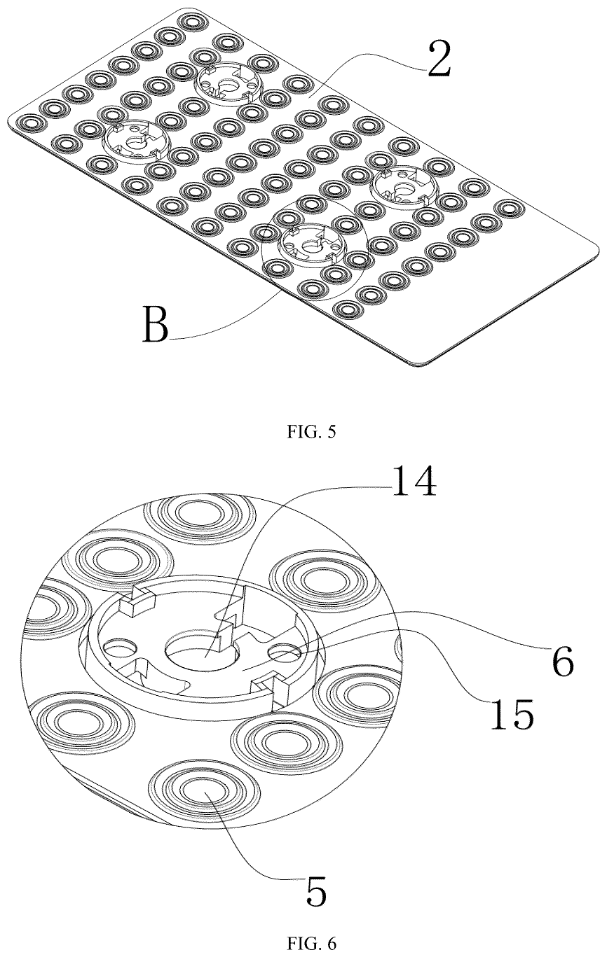

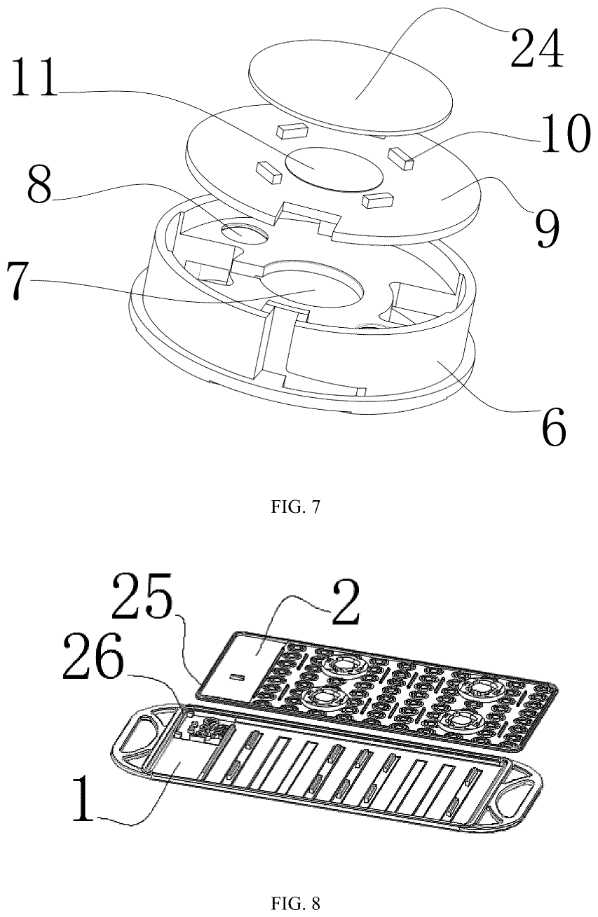

As shown in FIG. 1 to FIG. 8, the application provides a multifunctional belt, which comprise a belt surface layer 1 and a belt inner layer 2, wherein the belt surface layer 1 is provided with a groove 26, the belt inner layer 2 is provided with a bulge 25 fitting with the groove 26, the belt inner layer 2 is buckled on the belt surface layer 1 through the fit of the bulge 25 and the groove 26, the belt surface layer 1 is provided with an LED light panel 3, the LED light panel 3 is provided with a plurality of LED lights 4, the belt inner layer 2 is provided with convex lenses 5 corresponding to the LED lights 4, that is, one LED light 4 corresponds to one convex lens 5, the convex lenses 5 and the LED lights are arranged up and down on the same axis, and light beams emitted by the LED lamps 4 irradiates to the skin through the convex lenses 5; the LED lights 4 are energized during phototherapy, so that light beams (which can be red or near-infrared light, etc.) emitted by the LED lights 4 focus on the surface and deep layer of the skin through the convex lenses 5 to improve the phototherapy effect; the light beams focus through the convex lenses 5, so that the belt has ingenious structure, good effect and low cost.

Preferably, the belt inner layer 2 is provided with a base 6, the base 6 is provided with a vibrating motor 7, vibratory massage is performed on the human body through the vibratory motor 7.

Preferably, the base 6 is provided with a magnet 8, which can generate a certain magnetic field to perform magnetic therapy on the human body and promote blood circulation, and the magnet 8 is installed on the base 6, which makes reasonable use of space and expands the function.

Preferably, each base 6 is provided with a PCBA board 9, the PCBA board 9 is provided with a plurality of resistors 10 and energized for heating through the resistors 10, and the heat source emitted by the resistors 10 is used for thermal therapy on the human body; obviously, the PCBA board 9 and the resistor are installed on the base 6, and the space is reasonably utilized, thus reducing the belt volume and improving the belt function.

Specifically, the base 6 is provided with a stainless steel cap 601, the stainless steel cap 601 is covered on the base 6, all PCBA boards 9 are provided with EMS conducting areas 11, one half of which have positive electrodes, and the other half of which have negative electrodes; after the PCBA boards 9 are energized, current generated by the EMS conducting areas 11 is conducted to the human body for electrotherapy through the stainless steel cap 601, realizing massage and physiotherapy on the human body through current conduction and electrical stimulation; obviously, the PCBA boards 9 are located on the base 6, and the space is reasonably utilized, thus reducing the belt volume and weight; the stainless steel cap 601 is not easy to rust, and the surface is smooth and easy to clean.

Specifically, the belt surface layer 1 is provided with a grille 12, an inner frame of the grille 12 fits an outer frame of the LED light panel 3, the LED light 4 panel 3 is installed on the grille 12 to position the LED light panel 3, ensure that the LED light 4 and the convex lens 5 are on the same axis and prevent the LED light panel 3 from deviating, and the structure is simple and easy to install and maintain.

Specifically, the belt surface layer 1 and the belt inner layer 2 are made of silicone, which is soft to improve the comfort of use and has good thermal conduction effect.

Specifically, the base 6 is provided with a motor hole 14 and a magnet hole 15, and the vibrating motor 7 is installed on the motor hole 14, and the magnet 8 is installed in the magnet hole 15; the motor hole 14 and the magnet hole 15 are arranged to facilitate the installation of the vibrating motor 7 and the magnet 8 and play a limiting role to avoid shaking.

Specifically, the belt surface layer 1 is provided with a battery 23 and a PCB board 16, the battery 23 is connected with the PCB board 16 through a wire, the belt surface layer 1 is provided with a power switch 17, a heating switch 18, an EMS switch 19, an indicator light 20 and a charging port 21, all of which are connected with the PCB board through wires, the power switch 17 is used to control the on-off of the power supply and the on-off of the vibrating motor 7, the heating switch 18 is used to control the on-off of the heating function, the EMS switch 19 is used to control the on-off of the EMS conducting areas 11 to control the on-off of the microcurrent, the indicator light 22 is used to indicate the state of the belt, and the belt surface layer 1 is provided with a gland 22, which is used to cover the battery 23 and the PCB board to prevent shaking and damage.

Preferably, a silicone thermal conductive pad 24 is arranged on the resistor 10, and heat generated by the resistor 10 is transferred to the stainless steel cap 601 through the silicone thermal conductive pad 24 to reduce heat loss and avoid direct contact of the resistor 10 with the stainless steel cap 601, which is easy to cause damage.

The above is only the embodiment of the invention, but the scope of protection of the invention is not limited to this. Any changes or substitutions that any technicians familiar with the technical field can easily think of within the technical scope disclosed by the invention should be covered by the scope of protection of the invention. The scope of protection of the invention should be subject to the scope of protection of the claims.

1. A multifunctional belt, characterized by comprising a belt surface layer and a belt inner layer, wherein the belt inner layer is buckled on the belt surface layer, the belt surface layer is provided with an LED light panel, the LED light panel is provided with an LED light, the belt inner layer is provided with a convex lens, and the LED light emits a light beam to be radiated onto the skin through the convex lens.

2. The multifunctional belt according to claim 1, characterized in that the belt inner layer is provided with a base, and the base is provided with a vibrating motor.

3. The multifunctional belt according to claim 2, characterized in that the base is provided with a magnet.

4. The multifunctional belt according to claim 2 or 32, characterized in that the base is provided with a stainless steel cap, the stainless steel cap is covered on the base, the base is provided with a PCBA board, the PCBA board is provided with a resistor.

5. The multifunctional belt according to claim 4, characterized in that the PCBA board is provided with an EMS conducting area.

6. The multifunctional belt according to claim 1, characterized in that the belt surface layer is provided with a grille, on which the LED light panel is installed.

7. The multifunctional belt according to claim 1, characterized in that the belt surface layer and the belt inner layer are made of silicone.

8. The multifunctional belt according to claim 3, characterized in that the base is provided with a motor hole and a magnet hole, and the vibrating motor is installed on the motor hole, and the magnet is installed in the magnet hole.

9. The multifunctional belt according to claim 1, characterized in that the belt surface layer is provided with the battery and the PCB board, and the battery is electrically connected with the PCB board, and the belt surface layer is also provided with a power switch, a heating switch, an EMS switch, an indicator light and a charging port, all of which are connected with the PCB board through wires.

10. The multifunctional belt according to claim 4, characterized in that a silicone thermal conductive pad is arranged on the resistor.
